(19) United States
(12) Patent Application Publication
(10) Pub. No.: US 2010/0298906 A1
(43) Pub. Date: Nov. 25, 2010

(54) SYSTEM, METHOD AND APPARATUS FOR CONTROL OF ENTEROSTOMIES

(75) Inventors: Linda Elizabeth Laidaw, New South Wales (AU); Anthony Clyde Neason Stephens, New South Wales (AU)

Correspondence Address: JONES DAY, 222 EAST 41ST ST, NEW YORK, NY, 10017, US

(73) Assignee: Continence Control Systems International Pty Ltd, Gordon, NSW (AU)

(21) Appl. No.: 12/733,825

(22) PCT Filed: Sep. 22, 2008
(86) PCT No.: PCT/AU2008/001405

§ 371 Date: Jun. 17, 2010

(30) Foreign Application Priority Data
Sep. 20, 2007 (AU) 2007905156

(51) Int. Cl. A61N 1/36

(52) U.S. Cl. 607/40

(57) ABSTRACT

An apparatus for effecting the closure of a stoma, the apparatus comprising a stimulator arranged to provide a signal for stimulation of implanted contractile tissues positioned proximate to the stoma in order to facilitate closure of the stoma.

U.S. Pat. No. 6,659,936 issued on 9 Dec. 2003, International Patent Application No. PCT/AU00/00925 filed on 4 Aug. 2000, Australian Provisional Application No. AUPQ2026, filed on 4 Aug. 1999, relate to the control of continence. International Patent Application No. PCT/AU2005/001698 filed on 8 Nov. 2005 and Australian Provisional Application No. AU2004906393 filed on 8 Nov. 2004, relate to an implantable electrode arrangement.

International Patent Application

No. PCT/AU2006/001301 filed on 4 Sep. 2006 and Australian Provisional Application No. AU2005904830 filed on 2 Sep. 2005, relate to an implant for managing a medical condition.

Each of the above documents are incorporated herein by reference in their entirety.

### FIELD OF THE INVENTION

The present invention relates to a system, method and apparatus for controlling a stoma.

### BACKGROUND OF THE INVENTION

Stomas are openings which connect a bodily cavity to the outside environment. Stomas are typically artificially created (i.e. by a surgical procedure) and used for a variety of different medical reasons. In an enterostomy procedure, for example, a stoma is surgically created in a patient's abdominal region to create a passage to the small intestine to allow for drainage and/or for connection to a feeding tube. This may be necessary, for example, where disease has caused the patient to lose normal functioning of their small intestine.

Stomas may be connected to a collection device, such as a colostomy bag. The collection device collects bodily waste for subsequent disposal. Such collection devices may be uncomfortable to wear, susceptible to leakage, and are often quite noticeable, causing embarrassment and inconvenience to the patient. As a result, otherwise healthy individuals may be prevented from engaging in normal society.

To avoid having to wear a collection device at all times, a stoma plug may be placed over the stoma. However, stoma plugs are also susceptible to leakage and not entirely effective for preventing odors from escaping from the bodily cavity and may cause irritation to the stoma and surrounding skin.

### SUMMARY OF THE INVENTION

In accordance with a first aspect, the present invention provides an apparatus for effecting the closure of a stoma, the apparatus comprising a stimulator arranged to provide a signal for stimulation of implanted contractile tissues positioned proximate to the stoma in order to facilitate closure of the stoma.

The contractile tissue may be positioned about the stoma and may be formed as a sphincter positioned about the stoma.

The contractile tissue may be smooth muscle tissue.

The stoma may be surgically formed and may be connected to a portion of a patient's gastro-intestinal tract, such as the large intestine.

The stoma may be connected to a surgically formed pouch, wherein the surgically formed pouch is connected to a portion of the patient's gastro-intestinal tract.

The stimulator may be arranged to provide a different stimulation signal or no stimulation signal in order to allow the contractile tissue to relax to enable the stoma to open.

The stimulator may be arranged to provide a further signal for stimulation of the surgically formed pouch, wherein the stimulation causes a peristaltic-type movement of the pouch.

The stimulator may be arranged such that when the stoma is relaxed, the further stimulator provides the signal for stimulation of the surgically formed pouch when the stoma is relaxed, to effect the peristaltic-type movement, thereby facilitating emptying of the pouch.

The further stimulator may be arranged to provide a further signal for stimulation of the surgically formed pouch, wherein the stimulation causes a peristaltic-type movement of the pouch.

The further stimulator may provide the signal for stimulation of the surgically formed pouch when the stoma is relaxed, to effect the peristaltic-type movement, thereby facilitating emptying of the pouch.

The signal may be in the form of a pulse signal, arranged to maintain tone in the contractile tissue to maintain closure of the stoma.

The stimulator may be arranged to provide a different stimulation signal or no stimulation signal in order to allow the contractile tissue to relax to enable the stoma to open.

The stimulator may be an implantable stimulator and may be arranged to be implanted within a patient.

The apparatus may be in communication with or include a sensor arranged to sense an evacuation condition. On sensing an evacuation condition, the apparatus may initiate an alert. The alert may be a vibratory alert produced by a vibration module (for example, placed within the implanted stimulator). Moreover, in addition to, or in the alternative, the alert may cause the stimulation to cease, to thereby open the stoma and allow evacuation of the bowel.

In accordance with a second aspect, the present invention provides a device for effecting the closure of a stoma, comprising implanted contractile tissue positioned proximate to the stoma, and arranged to be stimulated to contract to facilitate opening and closing of the stoma.

In accordance with a third aspect, the present invention provides a controller for controlling a stimulator which is arranged to stimulate implanted contractile tissue positioned proximate to the stoma to facilitate closure of the stoma, the controller being arranged to provide a signal to the stimulator to vary the stimulation provided by the stimulator.

The controller may be arranged to provide a signal which causes the stimulator to vary the stimulation to the contractile tissue, resulting in the contractile tissue relaxing to allow the stoma to open.

The controller may also be arranged to provide a signal which causes the stimulator to provide no signal to the contractile tissue to enable the tissue to relax.

In accordance with a fourth aspect, the present invention provides a programmer for programming operation of a stimulator which is arranged to stimulate implanted contractile tissue positioned proximate to the stoma to facilitate closure of the stoma, the programmer including an interface enabling communication with the stimulator for programming of the stimulator.

In accordance with a fifth aspect, the present invention provides a system for effecting the closure of a stoma, the system comprising an apparatus in accordance with a first aspect of the invention, and a device in accordance with a second aspect of the invention.

In accordance with a sixth aspect, the present invention provides a system for effecting closure of a stoma, comprising an apparatus in accordance with a first aspect of the invention, and a controller in accordance with a third aspect of the invention.

In accordance with a seventh aspect, the present invention provides a method of effecting the closure of a stoma, comprising the steps of stimulating implanted contractile tissue positioned proximate to the stoma of a patient in order to cause the contractile tissue to contract, by way of providing a stimulation signal to an electrode arranged to transmit the signal to the contractile tissue.

In accordance with a eighth aspect, the present invention provides a method of effecting the closure of a stoma in a patient, comprising the step of implanting into the patient a stimulator device arranged to provide stimulation signals to implanted contractile tissue positioned proximate to the stoma in order to cause the tissue to contract to facilitate closure of the stoma.

The step of implanting the contractile tissue may comprise the step of implanting a contractile tissue sphincter about the stoma.

In accordance with a ninth aspect, the present invention provides a method of effecting the closure of a stoma, comprising the steps of implanting contractile tissue in a position proximate to the stoma, the contractile tissue being arranged to be stimulated to facilitate closure of the stoma to prevent the ingress or egress of fluid and/or solids from the stoma.

In accordance with a tenth aspect, the present invention provides an apparatus for effecting the closure of a stoma, the apparatus comprising a stimulator arranged to provide a signal for stimulation of contractile tissue through stimulation of nerve fibres innervating the contractile tissue, in order to facilitate closure of the stoma.

In accordance with an eleventh aspect, the present invention provides a device for effecting closure of a stoma, comprising innervated contractile tissue positioned proximate to the stoma, and arranged to be stimulated, through stimulation of nerve fibres innervating the contractile tissue, to facilitate closure of the stoma.

In accordance with a twelfth aspect, the present invention provides a controller for controlling a stimulator which is arranged to stimulate innervated contractile tissue positioned proximate to the stoma, through stimulation of nerve fibres innervating the contractile tissue, to facilitate closure of the stoma, the controller being arranged to provide a signal to the stimulator to vary the stimulation provided by the stimulator.

In accordance with a thirteenth aspect, the present invention provides a programmer for programming operation of a stimulator which is arranged to stimulate innervated contractile tissue to facilitate closure of a stoma, through stimulation of nerve fibres innervating the contractile tissue, the programmer including an interface enabling communication with the stimulator for programming of the stimulator.

In accordance with a fourteenth aspect, the present invention provides a method of effecting the closure of a stoma, comprising the steps of stimulating innervated contractile tissue positioned proximate to the stoma of a patient in order to cause the contractile tissue to contract, through stimulation of nerve fibres innervating the contractile tissue, by way of providing a stimulation signal to an electrode to arranged to transmit the signal to the contractile tissue.

In accordance with a fifteenth aspect, the present invention provides a method of effecting the closure of a stoma in a patient, comprising the steps of implanting into the patient a stimulator device arranged to provide stimulation signals to innervated contractile tissue in order to cause the tissue to contract, through stimulation of nerve fibres innervating the contractile tissue, to facilitate closure of the stoma.

In accordance with a sixteenth aspect, the present invention provides a method of effecting the closure of a stoma, comprising the steps of implanting innervated contractile tissue in a position proximate to the stoma, the innervated contractile tissue being arranged to be stimulated, through a stimulation of nerve fibres innervating the contractile tissue, in order to facilitate closure of the stoma to prevent the ingress or egress of fluid and/or solids from the stoma.

In accordance with a seventeenth aspect, the present invention provides an apparatus for effecting the closure of a stoma formed of innervated contractile tissue, the apparatus comprising a stimulator arranged to provide a signal for stimulation of the contractile tissue in order to facilitate closure of the stoma.

In accordance with an eighteenth aspect, the present invention provides a method of effecting a peristaltic-type movement in a surgically formed pouch, comprising the steps of stimulating implanted contractile tissue positioned proximate to the surgically formed pouch in order to cause the contractile tissue to contract, by way of providing a stimulation signal to an electrode arranged to transmit the signal to the contractile tissue.

In accordance with an nineteenth aspect, the present invention provides a method of effecting the closure of a stoma in a patient, comprising the step of implanting into the patient a stimulator device arranged to provide stimulation signals to a surgically created pouch connected to a stoma in order to cause the pouch to contract to induce a peristaltic-type movement.

In accordance with an eighteenth aspect, the present invention provides a device for effecting closure of a stoma, comprising a plug arranged to substantially seat within the stoma, and a stimulator, arranged to cause contractile tissue proximate to the stoma to contract, by way of providing a stimulation signal to an electrode arranged to transmit the signal to the contractile tissue.

In accordance with a twentieth aspect, the present invention provides a system for assisting in the evacuation of a portion of a gastro-intestinal tract, comprising a stimulator arranged to provide a signal for stimulation of implanted contractile tissues positioned proximate to the stoma in order to facilitate closure of the stoma, and a further stimulator arranged to provide a further signal for stimulation of the surgically formed pouch, wherein the stimulation causes a peristaltic-type movement of the pouch, wherein the stimulator and the further stimulator are arranged to be simultaneously operated, such that when the stoma is relaxed, the further stimulator provides the signal for stimulation of the surgically formed pouch, to effect the peristaltic-type movement, thereby facilitating emptying of the pouch.

### BRIEF DESCRIPTION OF THE DRAWINGS

Features and advantages of the present invention will become apparent from the following description of embodiments thereof, by way of example only, with reference to the accompanying drawings, in which:

FIGS. 1 & 1a are sagittal views through the bowel region of a patient illustrating an implanted system, in accordance with alternative embodiments of the present invention;

FIG. 2 is a sagittal view through the abdominal region of a patient illustrating an implanted system, in accordance with an alternative embodiment of the present invention.

FIG. 2a is a sagittal view through the abdominal region of a patient illustrating a partially implanted system, in accordance with an alternative embodiment of the present invention.

FIG. 3 is a block diagram of componentry of an implantable stimulator of the systems of FIGS. 1 to 3;

FIG. 4 is a block diagram of a system in accordance with an embodiment of the present invention;

FIG. 5 is a block diagram of a further system in accordance with an embodiment of the present invention;

FIG. 6 is a sagittal view through the abdominal region of a patient illustrating an implanted system, in accordance with yet another embodiment of the present invention.

FIGS. 7, 8 & 9 are exploded perspective, plan and side views, respectively, of an electrode arrangement for delivering stimulation signals in a system in accordance with an embodiment of the present invention;

FIGS. 10, 11, 12 & 13 are perspective, plan, side section, detail views of a shroud component of the electrode arrangement of FIG. 7;

FIGS. 15, 16, 17, 18, 19, are perspective, rear, plan section, side section and plan views of a cover component of the electrode arrangement of FIG. 7.

### DETAILED DESCRIPTION OF PREFERRED EMBODIMENTS

Embodiments of the present invention will hereafter be described in the context of a system, method and apparatus for controlling closure of a surgically created opening, or stoma. The stoma may be connected to a patient's small bowel and used to expel bodily waste. The stoma may be created in an enterostomy procedure, for example. However, it will readily be understood that embodiments of the present invention may equally be suited for controlling stomas connected to various other bodily cavities. For example, embodiments may be suited for controlling a stoma connected to a patient's large bowel or any other suitable site on the gastro-intestinal tract such as the stomach.

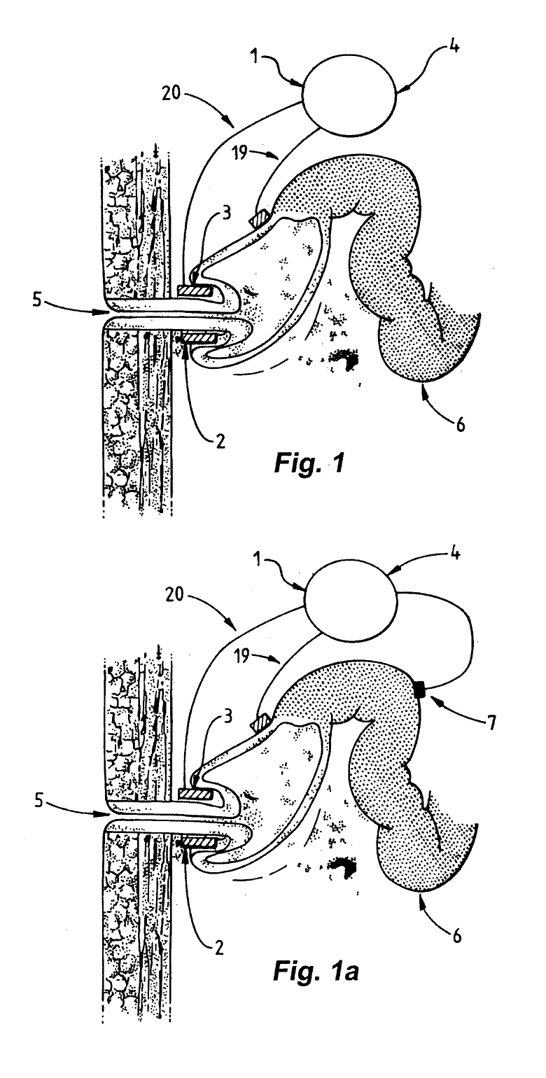

Referring to FIG. 1, a system and apparatus in accordance with an embodiment of the present invention, for controlling expulsion of bodily waste from a stoma, are illustrated in systematic form. The system includes an apparatus comprising an implantable stimulator 1 and a device comprising contractile tissue 2 which is arranged to be stimulated by a signal that is generated by the stimulator. In an embodiment, the signal is applied to the contractile tissue 2 which is formed into a neosphincter. The neosphincter 2 may be stimulated via an electrode 3 conductively connected between the stimulator 1 and contractile tissue 2.

In an embodiment, the stimulator 1 includes a signal generator for producing a pulsatile signal. The signal generator is housed in a bio-compatible housing 4 and will be described in more detail in subsequent paragraphs.

The neosphincter 2 in this embodiment is implanted surrounding the stoma in the patient's abdominal region to control the opening and closing of the stoma. In FIG. 1, the stoma is designated by reference numeral 5 and the small bowel by reference numeral 6. Stimulation of the contractile tissue neosphincter 2, in operation, causes the contractile tissue to contract and maintain closure of the stoma 5 to prevent expulsion of bodily waste. In other words, the contractile tissue 2 can be controlled to advantageously keep the stoma closed until evacuation of the small bowel is required.

It will be understood that the stoma could include a neosphincter formed from smooth muscle tissue and may be arranged to remain in a substantially “closed” configuration. The neosphincter, in turn, may be provided in any one of a number of configurations, including a naturally “open” position, where the neosphincter remains relaxed and the stoma remains open until such time as the neosphincter is stimulated. Alternatively, the neosphincter may be formed to ensure that the stoma remains, without stimulation, in a partially closed or completely closed position. The advantage of providing a naturally closed stoma (i.e. a naturally “tight” neosphincter) is that less electrical stimulation is required to keep the neosphincter in a closed position. It will be understood that embodiments of the invention may be used with either open or closed neosphincters. In an open neosphincter system, the stimulator would be utilized to effect closure of the stoma by virtue of a contraction of the neosphincter. In a closed stoma configuration, the stimulator, utilizing a smaller or lower level of stimulation, may be used to further “tighten” the neosphincter, to prevent leakage, rather than to effect closure of the stoma, per se. It will be understood that in the ensuing description, when reference is made to the stoma, neosphincter and the stimulator, it is intended to include variations in the abovementioned arrangement of the stoma, neosphincter and stimulator.

FIG. 1a shows an alternative embodiment. In the FIG. 1a drawing, the same reference numerals have been used as in FIG. 1 for equivalent components. Those components have the same function as in FIG. 1 and no further description will be given here. In such an embodiment, an additional electrode 7 is placed on, in or around the bladder to detect the onset of peristalsis. On detecting the onset of peristalsis, the electrode 7 sends a signal to the implantable stimulator 1 which in turn instructs the sphincter 2 to relax, thereby allowing the evacuation of the bodily waste out of the stoma 5. In an embodiment, the stoma 5 may be connected to a colostomy bag for collecting the evacuated waste. In an embodiment the lead/electrode may be bipolar, monopolar or multipolar.

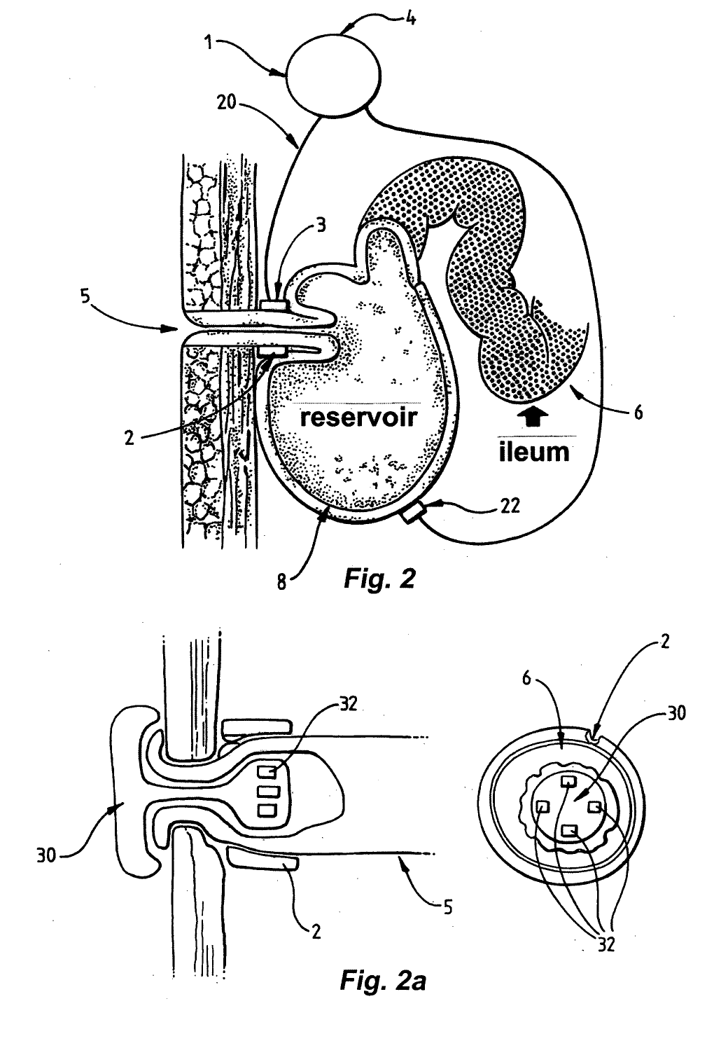

Yet another alternative embodiment is shown in FIG. 2. Again like numerals are provided to indicate equivalent components. In this embodiment, a surgically implanted internal reservoir 8 sits between the opening in the small bowel 6 and stoma 5. The internal reservoir 8 is created from intestinal tissue and may be implanted during an ileostomy, or “kock”, procedure. In FIG. 2, in addition to an electrode 3 for stimulating the sphincter 2 the implantable stimulator also connects to a reservoir electrode 22 for causing the reservoir to contract (i.e. affecting a peristaltic-type action). In an embodiment, the sequencing of the evacuation stimulation pattern is to first send a signal to electrode 22 for causing the reservoir to contract, then a second signal to the electrode 3 causing the sphincter 2 to relax. In another embodiment (not shown), a sensing lead is connected to the internal reservoir 8 to determine a direct or indirect measure of bladder volume, for indicating when drainage of the reservoir is required. Alternatively, where no sensory drive or feedback can be obtained (e.g. where the patient has sustained a spinal cord injury), the stimulator 1 may initiate an evacuation sequence in a timed manner. In an alternative embodiment, the signal for controlling the internal reservoir may be provided by a second implanted stimulator which is independent to the stimulator 1. In a further alternative embodiment, the implanted stimulator may be controlled by the presence of a stoma cap—so that when the stoma cap is placed in the stoma by the user, the stimulator is activated (maintaining tone), and when the stoma cap is removed, the stimulation is stopped (or more generally, changes to a different control signal) which causes the stoma to relax and promote emptying. Various means such as a magnetic reed switch could be used to cause the presence of the stoma cap to modify the implanted stimulator's status.

FIG. 2a shows an alternative embodiment where the stimulator is not implanted but rather provided in a stoma plug 30 for positioning within the stoma 5. In such an embodiment, one or more electrodes 32 are positioned about the plug 30 which can deliver signals to the electrode(s) 3 positioned on the neosphincter 2 for causing the neosphincter 2 to contract or relax. The electrode(s) 32 may be powered by a battery provided on the plug 30, or alternatively from a separate power source connected to the plug 30. In an embodiment, in use, the electrodes 32 may be constantly “on” causing the neosphincter 2 to be in a constant state of contraction whilst ever the plug 30 is positioned within or in close proximity to the stoma 5.

In an embodiment the plug 30 may additionally incorporate a gas absorption device such as an active charcoal chamber, or the like, for absorbing malodorous odors emanating from the bodily cavity 6 connected to the stoma 5. The gas absorption device may be disposable or reusable.

In an embodiment, the stoma may be formed of contractile tissue and direct stimulation of the stoma itself may directly control opening and closing of the stoma.

In an alternative embodiment, the stoma may be connected to a patient's gastro-intestinal tract. In such an embodiment, the sphincter may be controlled so as to allow for insertion of a feeding tube when evacuation of the gastro-intestinal tract is required. Once the evacuation procedure or feeding procedure has been completed, the sphincter may be stimulated to cause the contractile tissue to contract, thereby preventing any expulsion of gastro-intestinal matter. This may advantageously allow feeding tubes to be inserted as required, rather than permanently left in situ.

In another embodiment the contractile tissue is used for a urinary stoma where a catheter is inserted only when drainage is required and/or where a urinary collection system is used.

In an embodiment the contractile tissue is smooth muscle tissue. The smooth muscle tissue may be obtained from elsewhere in the body, formed into a sphincter and surgically implanted. Alternatively, the smooth muscle tissue may be grown from smooth muscle stem cells and/or proliferative smooth muscle cells. Alternatively, the smooth muscle tissue may be transplanted smooth muscle tissue augmented by smooth muscle stem cells and/or proliferative smooth muscle cells. Alternatively, the smooth muscle tissue may be the tissue of the internal fecal sphincter.

Alternatively, the smooth muscle tissue may be made from or include a man made substance, such as a polymer which contracts when an electrical current or signal is applied. One example, given here for illustration purposes only, is an “electroactive polymer”, which is a polymer that can contract or expand dependant on an electrical signal or current being applied to the polymer. Such polymers may be utilized to “augment” smooth muscle, or, alternatively, may be used in place of smooth muscle.

International Patent Application No. PCT/2006/001301, referred to above, discloses augmentation of contractile tissue using proliferative smooth muscle cells or smooth muscle stem cells. Growth, maturation and stability of the tissue may be influenced by growth factors (trophic and/or neuron-trophic factors) that are a component of the treatment.

Smooth muscle may be taken from other parts of the body or are grown (as discussed above). In an embodiment, the smooth muscle may be taken from the smooth muscle of the bladder and transplanted about the stoma, with its circulation intact. Alternatively, the muscle is venous smooth muscle, anococcygus smooth muscle or terminal ileum transplanted as a segment devoid of mucosa and having its circulation intact. A further alternative is the dartos smooth muscle from the scrotum or a portion of the vagina or labia.

In an embodiment, smooth muscle may be taken as a free graft, the tissue is separated from its normal circulation and comes vascularised by ingrowth of blood vessels at the side of implant.

Referring back to FIGS. 1 and 2, the stimulator 1 includes a signal generator arranged to provide a stimulation signal for stimulating the smooth muscle sphincter 2. A lead 20 extends from the stimulator 1 to the electrode 3 at the smooth muscle sphincter 2, providing the stimulation signal to the smooth muscle sphincter 2. The stimulation signal may be a signal of frequency and amplitude determined to maintain contraction of the smooth muscle sphincter 2 to facilitate continence.

The stimulator 1 may also be arranged to produce a further electrical signal to stimulate the sphincter 2 to relax, to allow the ingress or expulsion of matter into/out of the stoma 5. As an alternative to a further electrical signal, the stimulator 1 may be arranged to stop produce any electrical signal, and it is the absence of the signal that causes the sphincter 2 to relax. In this embodiment, the stimulator 1 is arranged to have the stimulation signal varied under control of the patient by way of external controls.

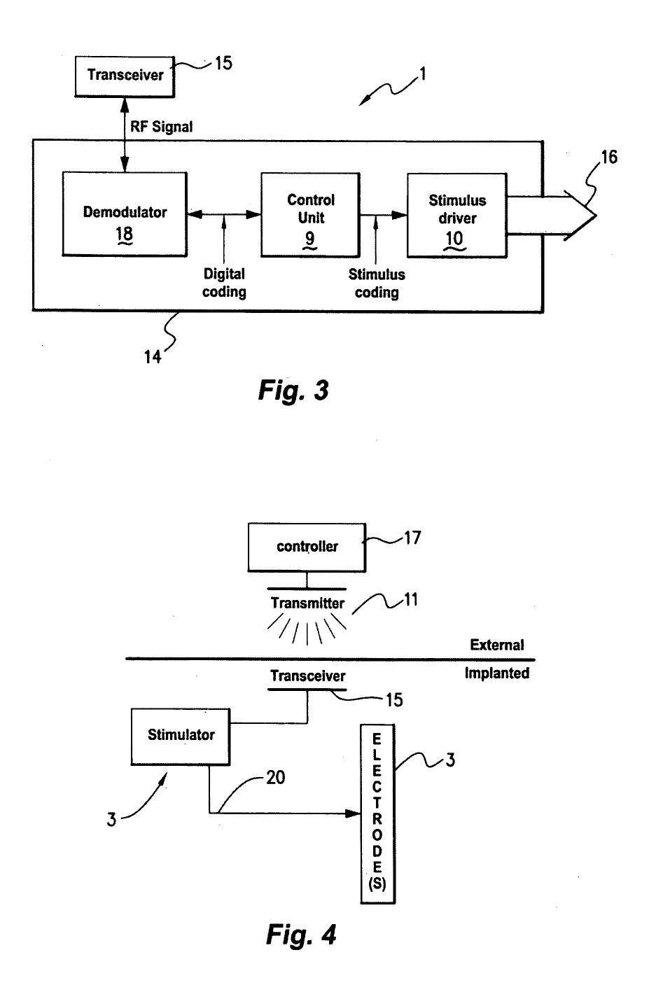

As described above, the stimulator 1 is also capable of sending stimulation signals to other bodily parts. For example, the stimulator 1 may send similar stimulation signals to the electrode 22 of the surgically implanted reservoir 8 (as illustrated in FIG. 3) for causing the internal reservoir to contract or relax.

The stimulator 1 is shown in more detail in FIG. 3. In this embodiment, a signal generator that is arranged to provide the electrical signal for stimulation of the sphincter 2 is in the form of a control unit 9 and stimulus driver 10. The control unit 9 encodes the stimulus and provides a signal to the stimulus driver 10 which provides the stimulation signal at output 16. The output 16 outputs to conductor 20 and to one or more electrodes 3 and 22.

In this embodiment, the control unit 9 and stimulus driver 10 form, together with a demodulator 18, a processing unit for generating the stimulation signal(s) at output 16.

The demodulator 18 is arranged to demodulate a signal received by transceiver 15. An external control unit and external programmer unit (both to be described later) are able to communicate via the transceiver 15 with the processing unit 14 in order to control application of stimuli and/or vary the stimuli. In addition, as described in more detail later, the processing unit 14 may transmit, via control unit 9, demodulator 18 and transceiver 15, signals to the control unit or programmer unit. The transmitted signals may deliver telemetry information indicative of parameters of the stimulator, for the purposes of calibration and control. The transceiver 15 may also be operable to receive a signal indicative of when a contraction/relax signal should be output. For example, the transceiver 15 may receive a signal from the reservoir sensor in FIG. 3 which will provide an indication as to the level of fluid in the reservoir. Based on this indication, the processing unit 14 may decide to instruct an evacuation or closure process.

The entire stimulator 1 (including components 14 and 15), is enclosed in a housing which includes a casing made from a bio-compatible material, such as titanium, silicone polymer other acceptable materials, or combinations of materials, including, but not limited to inert materials. The frequency of the RF signal for transmission and reception by the transceiver 15 may depend on the material of the casing of the stimulator.

FIG. 4 shows a system in accordance with an embodiment of the present invention. The system incorporates the implanted stimulator 1, with transceiver 15. The electrode(s) 3 is shown schematically together with cable 20. The system also comprises an external controller 17 which includes a transmitter 11. The controller 17 is intended for operation by a patient with the stimulator implanted, for control of the stimulator 1. The controller 17 includes an actuator (such as a button, not shown) operable by the patient to selectively send signals to the implanted stimulator 1, for control of the stimulation signals being sent to the electrode(s) 3.

In one embodiment, the stimulator is “fail safe”. Unless a signal is received from the controller 17, the stimulator produces a signal which maintains tone in the smooth muscle implant 2, maintaining closure of the stoma.

When the patient wishes to open the stoma, the patient actuates the controller 17 to send, via the transmitter 11, a signal to the stimulator 1. In response to receiving the signal, the control unit 9 operates to turn the stimulating signal off causing the sphincter 2 to relax and allow the bodily cavity to be open to the outside environment.

The controller 17 may also be arranged to provide a further signal under patient control, once the patient wishes to close the stoma (e.g. when a feeding tube, colostomy bag etc have been removed), the further signal causing stimulator 1 to resume providing the stimulation signals to the electrode(s) 3.

In “fail safe” mode, if the further signal is not produced, the stimulator may resume providing the stimulation signal to the electrode(s) 3 after a predetermined period of time.

It will be understood that the controller may also be in communication with an appropriate physiological sensor 21, which senses, either directly or indirectly, need for a patient to evacuate their bowels, by for example, measuring the amount of volume/fluid contained within the colon and/or bladder, or detecting the onset of peristalsis.

That is, the sensor is arranged to detect an evacuation condition. The evacuation condition may be measured in a number of manners.

Where a direct measurement technique is utilized, the sensor may take the form of a pressure sensor, for example, arranged to measure the pressure in the bowel/bladder or at the stoma/neosphincter interface. The sensor, upon detecting a full or almost full colon/bladder may provide a warning signal to the patient, to alert the patient of the need to evacuate their bladder/bowel. The alert may take the form of a vibration, an audible signal or a visual warning signal (or any combination thereof) which may emanate from the controller 17, or, in some cases, directly from the stimulator 1.

Where an indirect measurement technique is utilized, the sensor may measure electrical signals in or movement of the bowel (such as the onset of peristalsis), which may be an indicator that the bowel is full, or that the bowel requires evacuation.

The system may include a set of emergency instructions, such that if the patient does not evacuate their bladder/bowel, the system may take steps to prevent physical damage occurring to the neosphincter, the stimulator and/or the patient's bladder/bowel. For example, the system may be a closed loop system, arranged to automatically open the neosphincter after a given period of time, if opening is not voluntarily initiated by the patient.

Alternatively, where such a system is not appropriate, an open loop system may be employed, where no action is taken unless a specific command is received from the patient. However, in such a system the alert issued by the system becomes more frequent as the bowel/bladder continues to fill.

The stimulation signal 16 provided to contract the smooth muscle sphincter 2 is selected so as to provide a substantially continuous tone in the sphincter 1. The signal may be a generally rectangular and symmetrically biphasic pulse may be suitable for providing the substantially continuous tone. The signal has a substantially constant current less than or equal to 50 mA, 15 mA, 10 mA, or 5 mA, and in some preferred embodiments may be in the order of 4 mA, 8 mA, 12 mA, or 15 mA.

Stimulation pulse frequency provided to sphincter 1 is in the range of 0.1 Hz to 5 Hz, 0.2 Hz to 4.0 Hz. 0.25 Hz to 3.0 Hz, 1 Hz to 3.0 Hz, 1.5 Hz to 3 Hz, 1.75 Hz to 2.5 Hz, or a 0.25 Hz to 2.25 Hz, and in one embodiment, is 1 Hz, 2 Hz, 2.5 Hz or 3 Hz. Stimulation phase width of each phase is in the range of 0.05 ms to 2.0 ms, 0.1 ms to 1.5 ms, 0.2 ms to 1 ms, 0.25 ms to 0.75 ms, and in one embodiment is 0.2 ms, 0.4 ms, 0.5 ms or 1 ms. The stimulator is current regulated, and accordingly the stimulation voltage will vary with the resistance of the muscle tissue between the electrodes.

Typical values for the voltage are between 0.1 and 15 Volts, 0.2 and 12 Volts, 0.5 and 12 Volts, 0.5 and 10 Volts, or 0.5 and 7.5 Volts. In one embodiment, the voltage is 2.5 Volts, 5 Volts, 7.5 Volts or 10 Volts. Either a current source (voltage limited) or a voltage source (current limited) stimulator may be used.

It is also possible to use an asymmetric biphasic pulse, in which, for example, the first phase is shorter in duration than the second phase.

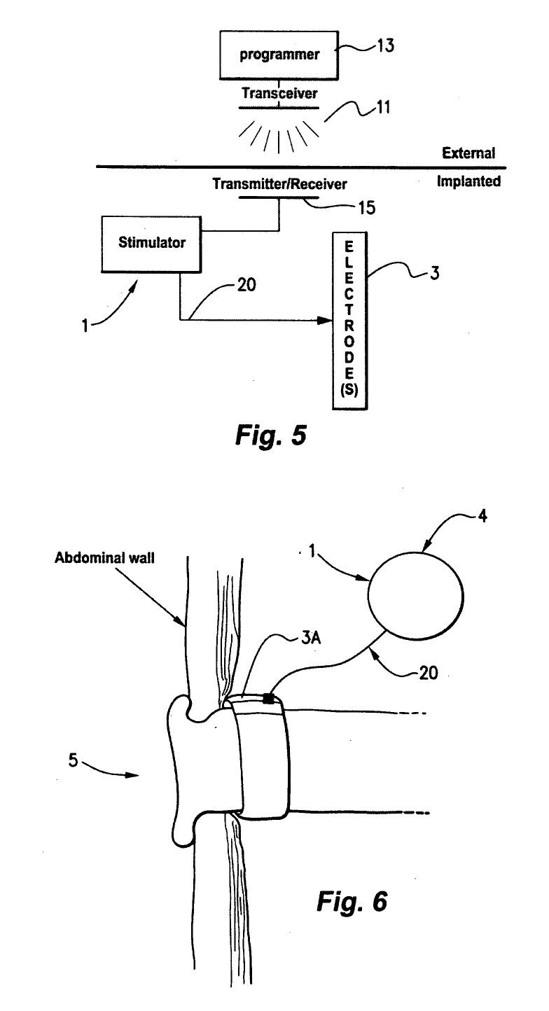

FIG. 5 shows a system in accordance with an embodiment of the present invention, including a programmer unit 13 which may be utilised by a physician to set and adjust parameters of the implanted stimulator 1. The programmer unit is arranged for communication with the stimulator via transceiver 11, and may comprise a computing device. The control unit 9 is also arranged to transmit stimulator telemetry information indicative of one or more of the parameters of the stimulator 1, for detection by the programmer 13 via transceiver 11. The programmer unit 13 can therefore determine parameters of the stimulator from telemetry information and can adjust the parameters by transmitting control signals to the stimulator 1. The signal from the programmer may be able to selectively vary the output current, shape, frequency and/or pulse width of the stimulation signal(s).

In operation, a physician adjusts parameters of the stimulation signal(s). The physician will note feedback from the patient as to the effect of the stimulus on expulsion control, for example, and may subsequently re-adjust the parameters until the stimulation is optimum. For example, patient perceived feedback may be used to set the maximum stimulation threshold of the smooth muscle sphincter.

In an embodiment, the control unit 9 may be provided with onboard memory for to collect event data including when and what type of stimulation signal was sent to the neosphincter for data logging purposes. The event data may be provided via transceiver 11 to the programmer 13 for inspection by a physician so as to better understand the patient's evacuation or feeding habits, etc.

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller 17 to cause the stimulator to stop producing stimulation signals and therefore allow the patient to defecate. In this embodiment, the controller 17 may be a simple magnet which, when passed over a magnetic receiver of the stimulator 1, results in the stimulator ceasing to provide stimulation signals for contracting the sphincter.

Other means than magnetic transmission may be utilised.

In the above embodiments, any suitable electrode(s) may be utilised to stimulate the implant 2. For example, button electrodes, cuff electrodes or any other suitable electrode may be utilised.

In embodiments, an electrode arrangement such as the arrangement disclosed in PCT/AU/20054/001698 may be utilised.

FIG. 6 illustrates an embodiment of the present invention where a “peg” electrode 3A such as disclosed in PCT/AU20054/001698 is utilised to transmit signals to the neosphincter 2 from the stimulator 1.

In FIG. 6, the same reference numerals as used in previous embodiments have been utilised to designate similar components, and no further description will be given here of these components.

The electrode 3A will now be described in more detail.

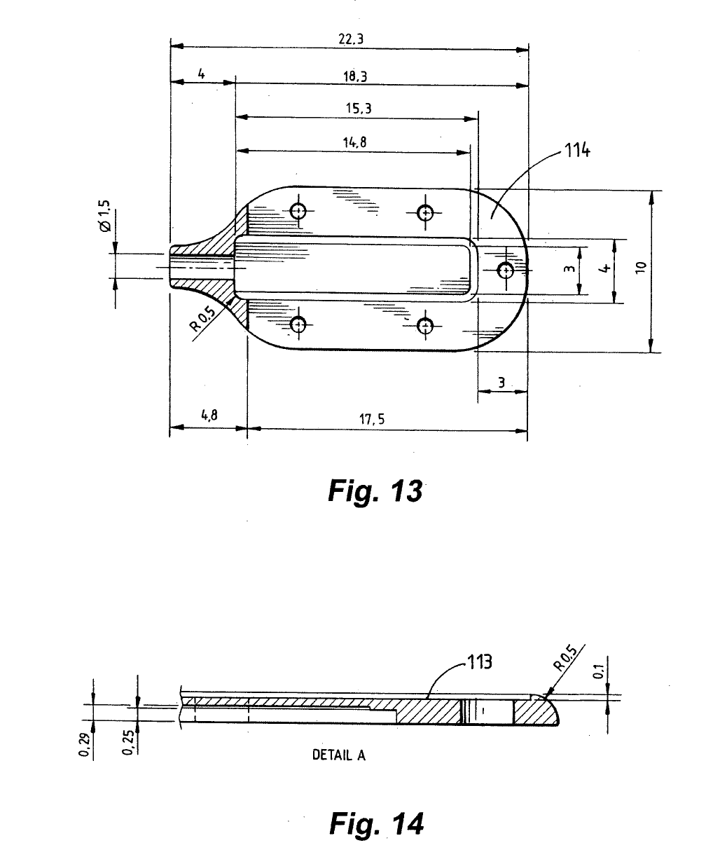

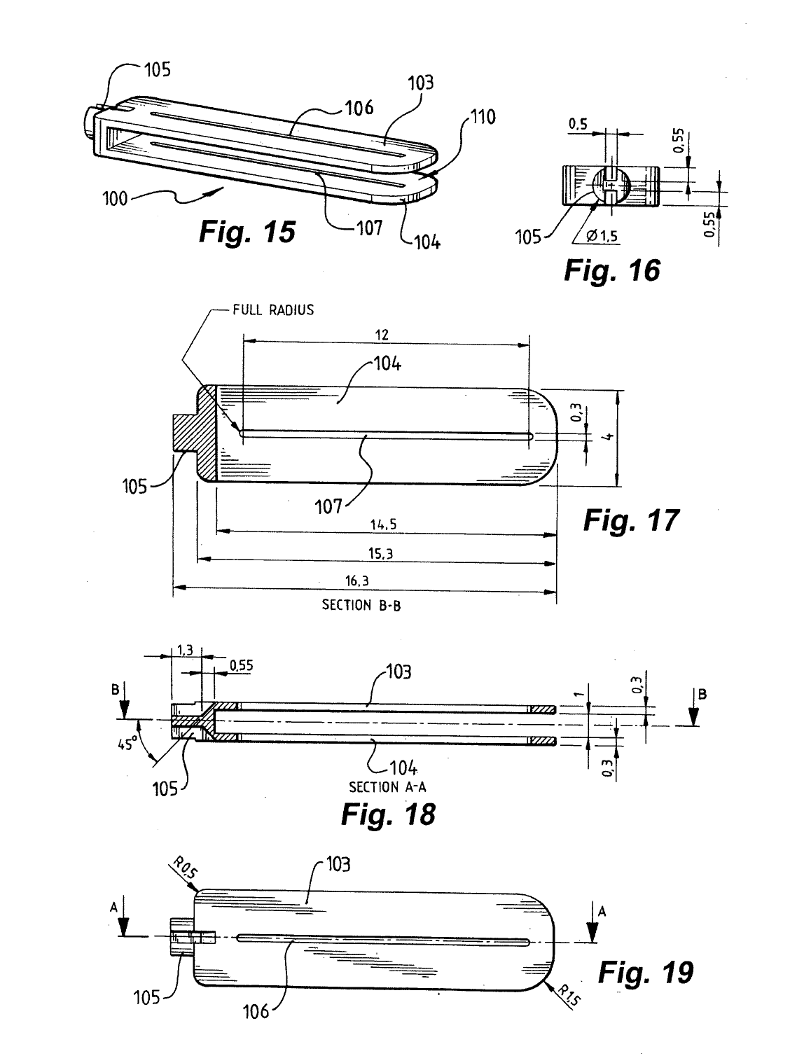

The electrode comprises a number of components. These include an electrode cover 100 (shown in most detail in FIGS. 14 through 19).

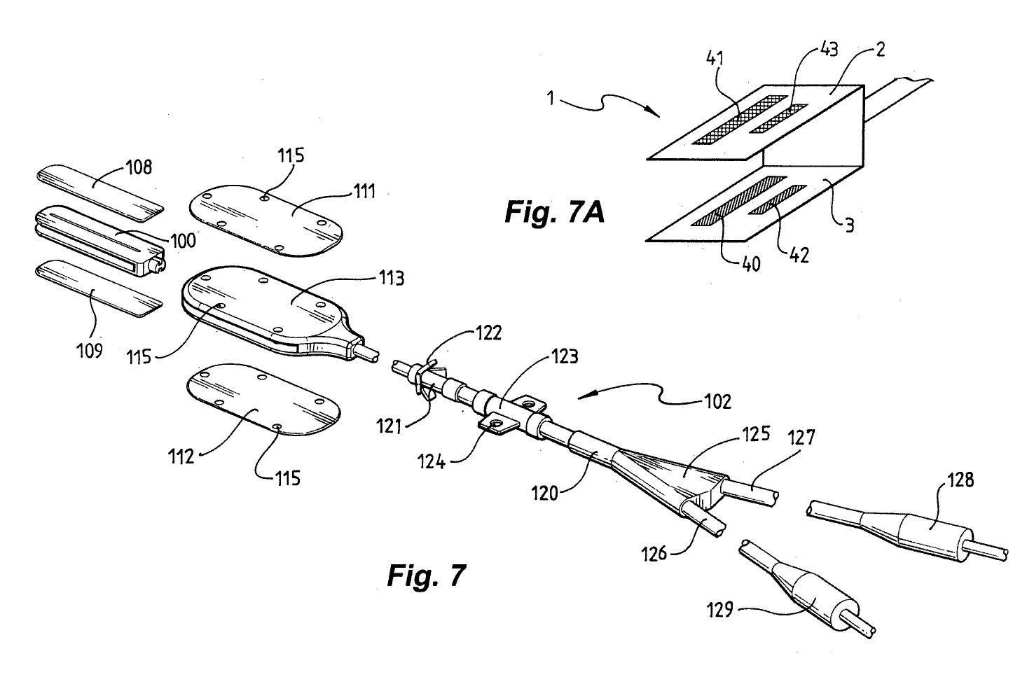

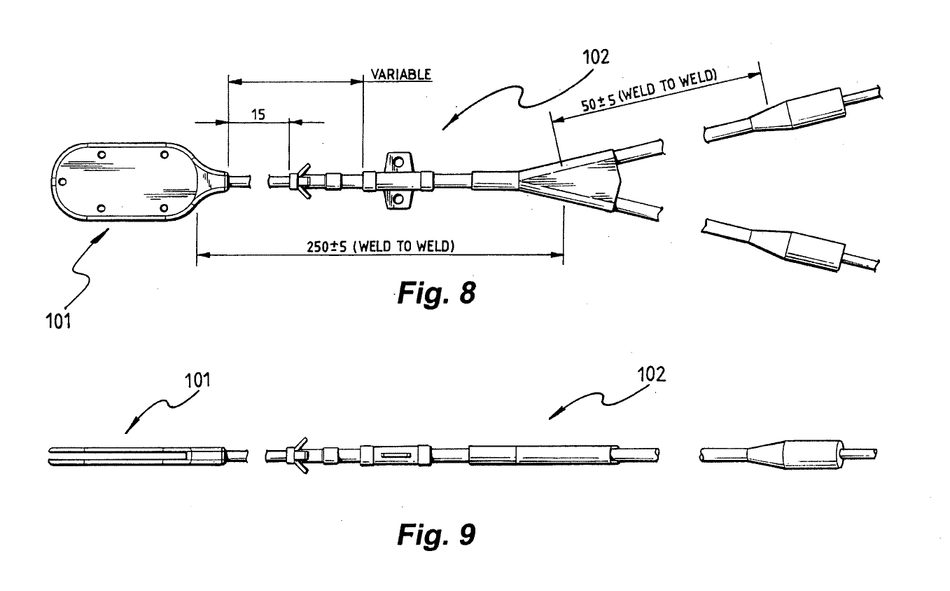

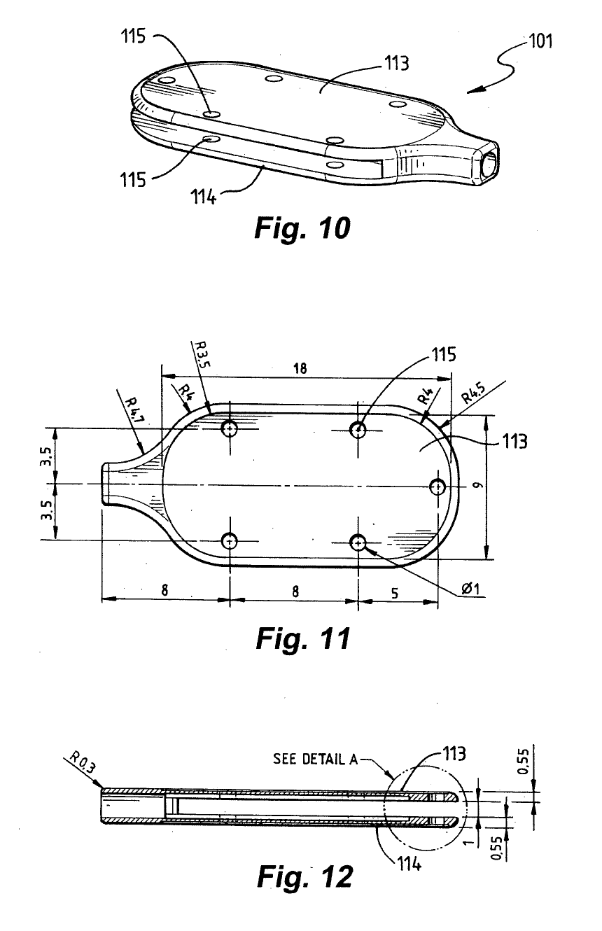

The components also include an electrode shroud (shown in best detail in FIGS. 10 through 13) and also an electrode lead 102 (shown in FIGS. 7, 8 & 9, together with the other components of the electrode arrangement).

In this embodiment first and second electrode elements are formed by the electrode cover 100, which includes insulating elements 103,104 extending from a base 105. The insulating extending elements 103,104 are formed with a slot 106,107, respectively, extending substantially along the length of the extending elements 103,104. When the electrode arrangement is assembled, platinum foil electrodes 108,109 (FIG. 7) are placed on the outer surfaces of the elements of the elements 103,104 so that they are insulated from the gap 110 formed between the elements 103,104 apart from the slots 106,107, which expose portions of the conductive plates 108,109 to the gap 110 (and, in use, to any tissue seated within the gap).

When assembled, the electrode cover 100 and platinum electrode foils 108,109 seat within the electrode shroud 101 as best shown in FIGS. 10, 11, 12 & 13. FIG. 14 in particular shown in cross-section where the electrode cover seats.

Electrode shroud 1 is formed from silicone. In order to provide reinforcement, PET mesh covers 111,112 are provided to fit to upper 113 and lower 114 extending portions of the shroud 101. Suture holes 115,116 are provided in the covers 111,112 and also in the elements 113,114 of the shroud 101. Note that the reinforcement can be provided by other means and is not limited to PET mesh. Further, the electrode shroud need not be in silicone but could be of other bio-compatible material and may not require re-enforcement. Further, note that other means for affixing to the tissue may be provided other than suture holes or instead of suture holes.

The electrode lead 102 is a multi-component arrangement which includes an outer insulating cover 120, a tine collar 121 including tines 122 for retaining the lead in position within a patient. It also includes a sutured collar 123 including suture holes 124 for suturing to patient tissue to also facilitate retaining the lead 102 in position. There is also bifurcation moulding 125 which enables the lead to split into two parts 126,127 which may contain separate conductors, and connectors 128,129 which may be arranged to contact to a simulation device.

In the above embodiments, the electrode arrangement includes a pair of electrode elements which extend away from a base which joins them together at their proximal ends. In a further embodiment, a single electrode element which is not joined at any base is provided. This single electrode element may be used to provide stimulation to contractile tissue on its own, or may be used together with one or more similar electrode elements to provide stimulation.

In the above described embodiments, each electrode element is provided with a single electrode. The single electrode is an elongate electrode extending substantially the majority of the length of the electrode element.

One advantage of having thin electrodes bounded by insulating material on either side is that the arrangement operates to confine the electric field produced by the electrode to the tissue immediately adjacent the electrode. This reduces or prevents stimulation of tissue that it is not desirable to stimulate e.g. tissue external to a contractile tissue sphincter being controlled.

In operation, the electrodes 108, 109 and extending elements 103, 104 are positioned either side of the smooth muscle implant to enable signals to be transmitted to the implant for operation.

Electrode arrangement 3A allows application of an electric field between the opposing electrode elements to stimulate the tissue between them. The electric field in one embodiment is confined so that stimulation is to a band of tissue between the electrodes.

In one embodiment, innervation runs within the implant 2 perpendicular to the band of tissue being stimulated.

The elements in electrode 3A extend over the tissue in a manner analogous to that of a clothes peg. The elements in electrode 3A extend over the tissue in a manner analogous to that of a clothes peg.

Other electrode patterns then a single line electrode on the surfaces of the elements may be utilised.

FIG. 8A discloses one alternate electrode pattern.

As discussed above, in an embodiment, the stimulator implant is preferably sealed and encased in a biologically inert material such as a biocompatible silicone material. Metallic electrodes and leads may be of platinum-iridium alloy. The connecting wires are, in one embodiment, insulated with a silicon coating.

The implant may be placed between the abdominal muscle and the skin.

In the above embodiment, the stimulator is a totally implantable device. In an alternative embodiment, the stimulator may not be implantable. The stimulator in this embodiment may comprise a stimulator device having similar componentry to that discussed above in relation to the embodiment of FIGS. 4, 5 and 6, but being arranged to be placed externally of the patient. In one embodiment, signals are coupled to electrodes placed within the patient in order to stimulate the contractile tissue. Coupling may be by way of inductively coupling the signals across the patient's skin to an internally positioned electrode arrangement. In another embodiment, part of the stimulator componentry may be placed outside the patient and part inside the patient.

In the above embodiments a single stimulation signal generator is used to provide the electrical signal. Other embodiments may use two or more signal generators.

Other embodiments may use two or more stimulators, which may be placed in different locations.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for affecting the closure of a stoma, the apparatus comprising a stimulator arranged to provide a signal for stimulation of implanted contractile tissues positioned proximate to the stoma in order to facilitate closure of the stoma.

2. An apparatus in accordance with claim 1, wherein the implanted contractile tissue is positioned about the stoma.

3. An apparatus in accordance with claim 1, wherein the contractile tissue is connected to an electrode, the electrode being connected to the stimulator.

4. An apparatus in accordance with claim 1, wherein the contractile tissue is formed as a sphincter positioned about the stoma.

5. An apparatus in accordance with claim 1, wherein the contractile tissue is smooth muscle tissue.

6-8. (canceled)

9. An apparatus in accordance with claim 1, wherein the stoma is connected to a surgically formed pouch, wherein the surgically formed pouch is connected to a portion of the patient's gastro-intestinal tract.

10. An apparatus in accordance with claim 1, the stimulator being arranged to provide a different stimulation signal or no stimulation signal in order to allow the contractile tissue to relax to enable the stoma to open.

11. An apparatus in accordance with claim 9, wherein the stimulator is arranged to provide a further signal for stimulation of the surgically formed pouch, wherein the stimulation causes a peristaltic-type movement of the pouch.

12. (canceled)

13. An apparatus in accordance with claim 9, further comprising a further stimulator arranged to provide a further signal for stimulation of the surgically formed pouch, wherein the stimulation causes a peristaltic-type movement of the pouch.

14. An apparatus in accordance with claim 13, wherein the further stimulator provides the signal for stimulation of the surgically formed pouch when the stoma is relaxed, to effect the peristaltic-type movement, thereby facilitating emptying of the pouch.

15. An apparatus in accordance with claim 1, wherein at least one of the signal and the further signal is in the form of a pulse signal, arranged to maintain tone in the contractile tissue to maintain closure of the stoma.

16. An apparatus in accordance with claim 1, the stimulator being an implantable stimulator and being arranged to be implanted within a patient.

17. An apparatus in accordance with claim 1, the apparatus being in communication with or including a sensor arranged to sense an evacuation condition.

18. An apparatus in accordance with claim 17, wherein the apparatus, on sensing an evacuation condition, initiates an alert.

19. An apparatus in accordance with claim 18, further including a vibrating module, arranged to provide a vibratory alert upon the initiation of an alert.

20. An apparatus in accordance with claim 18, wherein the alert causes stimulation to cease, to thereby open the stoma and allow evacuation of the bowel.

21. A device for affecting the closure of a stoma, comprising implanted contractile tissue positioned proximate to the stoma, and arranged to be stimulated to contract to facilitate opening and closing of the stoma.

22. A device in accordance with claim 21, wherein the contractile tissue is in the form of a sphincter positioned about the stoma.

23. A device in accordance with claim 21, wherein the contractile tissue is connected to an electrode, the electrode being connected to the stimulator.

24. A device in accordance with claim 21, wherein the contractile tissue is smooth muscle tissue.

25. A device in accordance with claim 21, wherein the stoma is surgically formed.

26. A device in accordance with claim 21, wherein the stoma is connected to a portion of the gastro-intestinal tract.

27. (canceled)

28. A device in accordance with claim 21, wherein the stoma is connected to a surgically formed pouch, wherein the surgically formed pouch is connected to a portion of the patient's gastro-intestinal tract.

29-46. (canceled)

47. A method of affecting the closure of a stoma, comprising the steps of stimulating implanted contractile tissue positioned proximate to the stoma of a patient in order to cause the contractile tissue to contract, by way of providing a stimulation signal to an electrode arranged to transmit the signal to the contractile tissue.

48-70. (canceled)

71. A system for assisting in the evacuation of a portion of a gastro-intestinal tract, comprising a stimulator arranged to provide a signal for stimulation of implanted contractile tissues positioned proximate to the stoma in order to facilitate closure of the stoma, and a further stimulator arranged to provide a further signal for stimulation of the surgically formed pouch, wherein the stimulation causes a peristaltic-type movement of the pouch, wherein the stimulator and the further stimulator are arranged to be simultaneously operated, such that when the stoma is relaxed, the further stimulator provides the signal for stimulation of the surgically formed pouch, to effect the peristaltic-type movement, thereby facilitating emptying of the pouch.
